(19) United States
(12) Patent Application Publication
(10) Pub. No.: US 2026/0354744 A1
(43) Pub. Date: Nov. 12, 2026

(54) METHOD AND DEVICE FOR DIAGNOSIS OF MEDICAL CONDITIONS

(71) Applicant: NANOSE MEDICAL LTD., Natanya (IL)

(72) Inventors: Orit MAROM ALBECK, Herzliya (IL); Osnat SELLA TAVOR, Kfar Kisch (IL)

(73) Assignee: NANOSE MEDICAL LTD., Natanya (IL)

(21) Appl. No.: 19/125,821

(22) PCT Filed: Nov. 2, 2023
(86) PCT No.: PCT/IL2023/051129

§ 371 Date: Apr. 30, 2025

Related U.S. Application Data
(60) Provisional application No. 63/382,141, filed on Nov. 3, 2022.

(51) Int. Cl. A61B 5/00; A61B 5/08

(52) U.S. Cl. CPC A61B 5/4244; A61B 5/082; A61B 5/4842; A61B 5/7282; A61B 2562/0285; A61B 2562/04

(57) ABSTRACT

The invention includes methods for exploring breath content for determining presence and concentration of markers of medical importance, including presence, onset and progression of Nonalcoholic Fatty Liver Disease and/or Barrett's Esophagus.

### TECHNOLOGICAL FIELD

The invention generally concerns methods and tools for exploring breath content for the purpose of determining presence and concentration of markers of medical importance.

### BACKGROUND

Fatty liver disease (steatosis) is a common condition caused by having too much fat build up in the liver. A healthy liver contains a small amount of fat. It becomes a problem when fat reaches 5% to 10% of the liver's weight. Nonalcoholic Fatty Liver Disease (NAFLD) is characterized by morphological and biochemical changes typical for alcoholic steatohepatitis, even in patients not consuming alcohol.

NAFLD is currently the most prevalent chronic liver disease in high-growth countries. The condition affects one in three adults and one in 10 children in the US. Proper dietary and pharmacological treatment are essential for preventing NAFLD progression which may develop serious liver sequelae, including liver failure and hepatocellular carcinoma.

NAFLD can be accompanied by mild lobular hepatitis, as well as nonalcoholic steatohepatitis (NASH). NASH includes early NASH without or with insignificant fibrosis, scirrhous NASH with significant fibrosis or advanced fibrosis, NASH with cirrhosis, and hepatocellular carcinoma (HCC). Cirrhosis is the end-stage condition of necroinflammation and fibrogenesis of the liver induced by chronic hepatic injury. Therefore, cirrhosis is the main risk factor predisposing to hepatocellular carcinoma (HCC).

Disease progression is often asymptomatic with 50% of the cases diagnosed at advanced stages when episodes of liver decompensation occur. HCC ranks as the third most common cancer-related death worldwide. Only patients diagnosed at the earliest stages can benefit from curative treatments and expect a 5-year survival of 50%-80%. Unfortunately, in the Western countries, as few as 30% of the cases are detected at early stages despite the establishment of semiannual surveillance protocols for the at-risk populations. These data indicate a pressing need for more efficient screening tests for the early detection of chronic hepatic injury and HCC.

Liver biopsy is the current standard for assessing NAFLD histologic lesions. This procedure is invasive, costly, and prone to complications, some minor, such as pain, others more severe with a recorded risk of death of 0.01%. Also, the NAFLD cohorts of individuals are large, therefore liver biopsy is not a practical and efficient tool for identifying those at risk of advanced fibrosis. In addition, a liver biopsy sample is only a very small piece of the liver, which can lead to incorrect staging if this sample is not representative of the rest of the liver. Thus, liver biopsy can lead to sampling error, which may result in either over staging or under staging of fibrosis; sampling error may occur in up to 25-30% of liver biopsies. Another limitation of liver biopsy is that different pathologists can interpret the same sample differently, which can result in discrepancies in liver disease staging.

### General Description

Breath biomarkers can be useful biomarkers in patients with Nonalcoholic Fatty Liver Disease (NAFLD). Differences between control and diseased subjects observed in several studies reflect differences in the activity of the CYP450 enzyme system, reduction of liver functional units, reduced portal blood flow, and impaired liver extraction capacity. Chronic hepatic conditions result in downregulation of the CYP450 system, including CYP2C9 and CYP2C19. The reduction of functional liver units is a consequence of replacement of parenchymal tissue with fibrotic tissue that also alters the vascular architecture.

While having a different etiology than fatty liver, Barrett's esophagus (BE) too is a precursor to the development of various cancers, mainly esophageal cancer, which early detection is highly desired, yet not available. Barrett's esophagus (BE) is a condition in which the typical squamous epithelium of the esophageal mucosa is replaced with columnar intestinal epithelium. BE is a known precursor to the development of esophageal adenocarcinoma (EAC), a malignancy with a dramatically increasing incidence over the past 40 years. The risk of EAC among patients with BE is estimated to be 30-125 fold greater than that of the general population. EAC, though uncommon, has a poor prognosis, and is associated with a 5-year survival rate of <20%.

Given the rising incidence of fatty liver (NAFLD) or BE and related conditions and poor early detection thereof, new screening modalities, that do not require endoscopy or invasive methodologies and which are immediate and substantially free of false positive/negative results have the potential to reduce costs and expand access to screening for fatty liver and BE.

The inventors of the technology disclosed herein have developed a novel point of care platform (or “bedside” or “near patient” analysis) for a noninvasive cost-effective early detection of subjects predisposed of suffering from fatty liver (NAFLD) or BE and which early diagnosis can help in preventing the development of life-threatening conditions which may directly or indirectly evolve from either condition.

The methodology of the invention is based on a novel device and methods of use which detect low and typically ignored concentrations of volatile compounds (VCs), such as volatile organic compounds (VOCs), that are emitted in an exhaled breath of a subject and which are indicative of metabolic processes or other pathways leading to or responsible for or associated with either fatty liver conditions or BE. The technology has demonstrated ability to detect extremely low concentrations of such VCs, being as low as several hundred parts per billion (ppb), suggesting occurrence or prevalence of certain biological processes indicative of disease evolution.

The technology disclosed herein also provides an ability to monitor the progress of any medical treatment or medical regimen in patients suffering from fatty liver or BE or conditions associated with either disease state, or to monitor progress of a medical treatment in a subject of the general population undergoing treatment for any acute or chronic medical condition.

Thus, in a most general aspect, the invention provides a noninvasive and cost-effective device and method for detecting, monitoring onset or presence of NAFLD or BE in a subject and further a device and method for determining progression of the disease in a subject suffering from either NAFLD or BE and optionally undergoing medical treatment.

The invention also provides a noninvasive and cost-effective device and method for detecting or monitoring a reduction in activity of CYP450, including CYP2C9 and CYP2C19, e.g., said reduction in activity may be used as an indicator for existence or onset of NAFLD or other fatty liver-related diseases.

A device of the invention is configured for collecting and measuring or determining a content profile, e.g., presence and concentration, of volatile compounds such as VOCs in a gaseous sample obtained from a subject suspected of e.g., (i) suffering from a decline or a reduction in CYP450 activity or enzyme concentrations/levels, or (ii) NAFLD or BE, or (iii) a subject who is predisposed for suffering from enzyme inactivity or declining enzyme concentration/levels or from either disease, or (iv) one who is generally monitored to detect development of such conditions.

As used herein, reference to fatty liver or NAFLD relates to the fatty liver disease (steatosis) that is a common condition caused by having too much fat build up in the liver.

This may be a result of Nonalcoholic Fatty Liver Disease (NAFLD) characterized by morphological and biochemical changes typical for alcoholic steatohepatitis, even in patients not consuming alcohol. Symptoms of NAFLD may include mild lobular hepatitis, as well as nonalcoholic steatohepatitis (NASH), with or without insignificant fibrosis, scirrhous NASH with significant fibrosis or advanced fibrosis, NASH with cirrhosis, and hepatocellular carcinoma (HCC). Within the context of the present invention, fatty liver also encompasses cirrhosis—the end-stage condition of necroinflammation and fibrogenesis of the liver—which may be regarded as a main risk factor predisposing to hepatocellular carcinoma (HCC).

The term thus encompasses cirrhosis resulting from a variety of causes, e.g., alcoholic uptake, NAFLD, viral hepatitis; and liver fibrosis.

Different from fatty liver, Barrett's esophagus (BE) is a condition in which the typical squamous epithelium of the esophageal mucosa is replaced with columnar intestinal epithelium. BE, too, is known to develop into a malignancy.

Reference to “NAFLD or BE” encompasses each of the diseases as independent and separate medical conditions to be diagnosed according to the invention, wherein each constitutes a separate embodiment, as disclosed herein.

Cytochrome P450 (CYP450) is a hemeprotein that plays a role in the metabolism of many materials. The metabolism occurs in many sites in the body, including the liver, intestinal wall, lungs, kidneys, and plasma. As the primary site of metabolism, the liver functions to detoxify and facilitate excretion of foreign chemicals by enzymatically converting lipid-soluble compounds to more water-soluble compounds. Many disease states can alter CYP450 activity. NAFLD is characterized by lipid vesicle accumulation in the liver, which induces severe steatohepatitis (NASH), fibrosis, cirrhosis, and hepatic carcinoma. The CYP450 enzyme is dysregulated in NAFLD, which reduces the ability of the enzyme to metabolize certain materials. This inactivity may be detected early on and may be used as a marker for determining onset of diseases relating to the enzyme inactivity, e.g., NAFLD, liver lipid vesicle accumulation, NASH, fibrosis, cirrhosis, and hepatic carcinoma.

According to a first aspect, the present invention provides a method for determining a reduction or a decline in CYP450 activity or level (concentration) in a liver of a subject, the subject being diagnosed with or is suffering from a disease or a condition or has a predisposition of suffering from a disease or a condition manifesting such a reduction or decline or which is caused by such a reduction or a decline, the method comprising:
a) determining a volatile compound (VC) profile in a breath sample collected from the subject; and
b) comparing the VC profile to a VC profile of a control, as defined herein, and/or optionally to a VC profile obtained from the subject at an earlier time point(s), as defined herein; wherein a VC profile different from the control VC profile is indicative of one or more of (1) presence of the disease or condition, (2) absence of the disease or condition, or (3) an improvement or worsening of the disease or condition.

In some embodiments, the disease or condition manifesting a reduction or a decline in the enzyme activity or level (amount, concentration) is NAFLD.

The invention further provides a method of determining presence, evolution (or development of early onset) and/or progression of a disease or a condition relating to or associated with CYP450 inactivity or enzyme level (amount or concentration), being for example NAFLD, in a subject, the subject being diagnosed with or is suffering from the disease or the condition or has a predisposition of suffering thereform, the method comprising:
a) determining a volatile compound (VC) profile in a breath sample collected from the subject; and
b) comparing the VC profile to a VC profile of a control, as defined herein, and/or optionally to a VC profile obtained from the subject at an earlier time point(s), as defined herein; wherein a VC profile different from the control VC profile is indicative of one or more of (1) presence of the disease or condition, (2) absence of the disease or condition, or (3) an improvement or worsening of the disease or condition.

Also provided is a method of determining presence, evolution (or development of early onset) and/or progression of a disease or a condition relating to or associated with CYP450 inactivity or enzyme level (amount or concentration), being for example NAFLD, in a subject, the subject being diagnosed with or is suffering from the disease or the condition or has a predisposition of suffering thereform, the method comprising: a) determining a volatile compound (VC) profile in a breath sample collected from the subject, the profile being indicative of presence of limonene; and
b) comparing the VC profile to a VC profile of a control sample obtained from a healthy subject (which may be the same subject or a different subject not suffering from the disease or condition);
wherein where the VC profile is different from the VC profile of the control sample demonstrates an increase in the amount of limonene is indicative of presence or onset of the disease or condition.

The invention further provides a method of determining presence, evolution (or development of early onset) and/or progression of BE in a subject, the subject being diagnosed or is suffering from the disease or the condition or has a predisposition of suffering therefrom, the method comprising:
a) determining a volatile compound (VC) profile in a breath sample collected from the subject; and
b) comparing the VC profile to a VC profile of a control, as defined herein, and/or optionally to a VC profile obtained from the subject at an earlier time point(s), as defined herein; wherein a VC profile different from the control VC profile is indicative of one or more of (1) presence of the disease or condition, (2) absence of the disease or condition, or (3) an improvement or worsening of the disease or condition.

The invention further provides a method of determining onset of a disease selected from NAFLD and BE in a subject, the method comprising:
a) determining a volatile compound (VC) profile in a breath sample or headspace of a biological sample collected from a subject suspected of having the disease but not demonstrating symptoms directly or indirectly associated with the disease; and
b) comparing the VC profile to a VC profile of a control, as defined herein, and/or optionally to a VC profile of a sample obtained from the subject at an earlier time point(s), as defined herein; wherein a VC profile different from the control VC profile is indicative of onset of NAFLD or BE or existence of the disease.

In some embodiments, the method is capable of detecting sub ppm levels (ppb levels) of a volatile compound(s) indicative of the onset or existence of the disease.

In some embodiments, the disease is NAFLD.

In some embodiments, the disease is BE.

In some embodiments, the method comprises
a) exposing a gaseous sample (a breath sample or a headspace sample, as defined herein) comprising volatile compounds (VCs) to a sensor responsive to interaction with the volatile compounds;
b) detecting/measuring an output signal received from the sensor correlating with an interaction between the VCs and the sensor; and
c) using a learning and pattern recognition algorithm to determine presence of a pattern of volatile compounds indicative of a reduction in one or both activity or level of CYP450 in the subject.

The detection method is an electronic measurement, wherein an electronic sensor is used. The signal generated at the output of the electronic sensor is used for making a measurement which may be processed to generate a finger print or a pattern, being the VC profile, indicative of the VC types, amounts, ratios, etc, present in the exhaled breath. The electronic sensor may be used to determine any change in the sensor surface or at the sensor vicinity; namely a change at the surface of the nanoparticles used in a sensor array or in their vicinity, as further disclosed herein. The change is measured in comparison to a virgin sensor not exposed to a VC of any type. The difference measured/detected (as compared to the virgin sensor) may be a difference in resistance, capacitance, charge density, surface energy and others.

In some embodiments, the method comprises
a) exposing a gaseous sample (a breath sample or a headspace sample, as defined herein) comprising volatile compounds (VCs) to a sensor responsive to interaction with the volatile compounds;
b) detecting/measuring an output signal received from the sensor correlating with an interaction between the VCs and the sensor; and
c) using a learning and pattern recognition algorithm to determine presence of a pattern of volatile compounds indicative of an active disease state or an onset of a disease state, wherein the disease state is NAFLD or BE.

In some embodiments, the method comprising:
a) exposing a gaseous sample being a breath sample or a headspace sample comprising volatile compounds (VCs) to a sensor responsive to interaction with the volatile compounds;
b) detecting/measuring an output signal received from the sensor correlating with an interaction between the VCs and the sensor; and
c) determining presence of a pattern of volatile compounds indicative of NAFLD or BE.

In accordance with methods of the invention, where the sample comprises VCs that are unique in pattern or profile (considering type of VCs, composition of VCs, amount of VCs, etc, as further defined herein) to VC profiles indicative of an active disease state or indicative of markers which indicate presence or onset of NAFLD or BE, or a reduction or a decline in CYP450 activity or level, or to VC profile of healthy subjects not suffering from either condition, such a sample will provide a determination or an indication of presence or onset of the disease state or condition. The unique pattern of VC indicative of any of the conditions may be determined using a learning and pattern recognition algorithm. This algorithm enables learning, dimensionality reduction, classification, regression, optimization and pattern recognition.

Non-limiting examples of algorithms which may be used include artificial neural networks, multi-layer perception (MLP), generalized regression neural network (GRNN), fuzzy inference systems (FIS), self-organizing map (SOM), radial bias function (RBF), genetic algorithms (GAS), neuro-fuzzy systems (NFS), adaptive resonance theory (ART) and statistical methods including, but not limited to, principal component analysis (PCA), partial least squares (PLS), multiple linear regression (MLR), principal component regression (PCR), discriminant function analysis (DFA) including linear discriminant analysis (LDA) or cluster analysis including nearest neighbor.

In some embodiments, the algorithm is LDA.

Using such algorithms or others known in the art, a VC profile may be regarded as significantly different from a control VC profile by presence or absence of one or more VCs indicative of the disease, concentration (or amount) of the one or more VCs, presence or absence of other VCs in combination, ratio amounts between the various VOCs and a change in the presence or amount of one or more VCs over time. The term “significantly different” as used herein generally refers to a quantitative difference in the concentration or level of each VC from the set or combinations of VCs as compared to the levels of VCs in control samples obtained from, e.g., individuals not having the disease. A statistically significant difference can be determined by any test known to the person skilled in the art. Common tests for statistical significance include, among others, t-test, ANOVA1 Kruskal-Wallis, Wilcoxon, Mann-Whitney and odds ratio. Individual samples (of unknown status) can be compared with data from the reference group (negative control). An increase or decrease in the level as compared to a control or reference value or mean control level or reference value, or a change, difference or deviation from a control or reference value, can be considered to exist if the level differs from the control level or reference value, by about 5 percent or more, by about 10 percent or more, by about 20 percent or more, or by about 50 percent or more compared to the control level or reference value. Statistical significance may alternatively be calculated as P<0.05. Methods of determining statistical significance are known and are readily used by a person of skill in the art. In a further alternative, increased levels, decreased levels, deviation, and changes can be determined by recourse to assay reference limits or reference intervals. These can be calculated from intuitive assessment or non-parametric methods. Overall, these methods calculate the 0.025, and 0.975 fractiles as 0.025*(n+1) and 0.975*(n+1). Such methods are well known in the art. The presence of a VOC marker which is absent in a control, is also contemplated as an increased level, deviation or change. The absence of a VC marker which is present in a control, for example, is also contemplated as a decreased level, deviation or change.

Various other algorithms are known in the art, which are disclosed, for example, in U.S. Pat. Nos. 6,411,905, 6,606,566, 6,609,068, 6,620,109, 6,767,732, 6,820,012 and 6,839,636, each of which being incorporated herein by reference.

In some embodiments, the method comprises obtaining a device comprising
a sample collecting chamber;
at least one sensor assembly comprising one or a plurality of sensing regions, wherein the assembly is in gaseous communication with said sample collection chamber;
a closed loop channel assembly for directing said sample from the sample collecting chamber to the at least one sensor assembly and for circulating said sample from the sample collecting chamber over the at least one sensor assembly over a period of time;
optionally means for circulating the sample, e.g., a pump; and
a pattern recognition analyzer (e.g., configured for real-time analysis of a VC profile derived from content of the sample);
wherein the method is carried out on the device.

As used herein, a “sensor responsive to interaction with the volatile compounds” is any sensor capable of generating a signal upon coming in contact with a volatile compound. The interaction and thus generation of a signal indicative of said interaction may be immediate or may be concentration dependent. Typically, the sensor used in devices of the invention is such which permits immediate signal generation indicative of the interaction. The sensor may be provided in a form of at least one sensor assembly that is selected to be responsive and interact with VCs which are characteristic of presence or absence of NAFLD or BE or a change in the disease state.

In some embodiments, the sensor is a sensor assembly.

In some embodiments, the at least one sensor assembly comprises one or more chemically sensitive sensors and a processing unit comprising a learning and pattern recognition analyzer configured for receiving sensor output signals and comparing the signals to a stored data. In some embodiments, the analyzer is selected to enable learning, dimensionality reduction, classification, regression, optimization and pattern recognition.

In some embodiments, the sensor or sensor assembly is selected to be responsive and interact with VCs characteristic of a disease state or onset of NAFLD or BE or a reduction in CYP450 activity or level.

In some embodiments, the sensor or sensor assembly is selected to be responsive and interact with NAFLD related VCs selected from: Limonene (D-Limonene), Isoprene, Alpha-pinene, α-terpinene, Styrene, Propene, 2-Nonene, 1-decene, 1-heptene, 1-octene, Benzene, 2-Butanone, 2-pentanone, Acetone, Acetophenone, 2-Octanone, Ethanol, Methanol, 2-Propanol, Indole, Dimethyl-sulfide, Hydrogen-sulfide, carbon-disulfide, Methylmercaptan, Methyl-amine, Trimethyl-amine (TMA), Methane, Ethane, Pentane, Octane, Tridecane, 3-methyl-hexane, acetic acid, propionic acid, Acetaldehyde and others.

In some embodiments, a sensor or a sensor assembly suitable for interaction with VCs according to the invention may be in a form of a functionalized surface region, a sensor having a functionalized nanowire or a nanotube, a polymer-coated surface acoustic wave (SAW) sensors, sensor employing a semiconductor gas sensor technology, aptamer biosensors, or amplifying fluorescent polymer (AFP) sensor.

In some embodiments, the sensor is or comprises nanoparticles.

In some embodiments, the nanoparticles are not of a metal oxide.

In some embodiments, the sensor is provided in the form of a plurality of nanoparticles associated to a surface. The sensor surface may comprise one or more sensing regions, each of the regions being associated with same or different population of nanoparticles, such that a signal may be independently derived from each of the sensing areas, and be indicative of an interaction (or lack thereof) between VCs present in the sample and the nanoparticles on the sensing regions.

Within the context of the present invention, volatile compounds (VOC) are compounds, typically but not necessarily volatile organic compounds (VOCs), that are associated with the metabolic processes or any other cellular process involved in the evolution or progression or development of NAFLD or BE. VCs that are generated in the body or are absorbed from the diet or inhaled in are released into the circulatory system and thereafter excreted through the skin, the urine, saliva blood and/or exhaled breath. The VCs may comprise a plurality of compounds, some of which are gaseous, others may be liquids (at a physiological temperature), which are released into the excreted biological sample or breath, and thus can be detected and quantified. When released via exhaled breath, the VCs may be carried by breath gases or small droplets of water.

The “VC profile” refers to a measured signature or an electronic signal pattern or electronic signature derived from a collection of properties relating to the VC content of the sample, e.g., exhaled breath, which is indicative of a disease state or onset, as disclosed herein. These collective properties are unique to samples obtained from diseased subjects and are thus informative, transformed into an electric signal or a fingerprint or a signature that can provide an indication of onset, evolution or progression of NAFLD or BE or a reduction in the activity of CYP450 due to inactivation or a reduction in its level. The VC profile can also provide an insight as to the state of the disease or the progression thereof, can identify the onset of the disease at an early stage before symptoms develop and can assist in determining success of a therapeutic treatment (prophylaxis or treatment of existing symptoms). The signal patterns derived from the collective properties may vary based on one or more of:
presence or absence of one or more VCs indicative of the disease,
the concentration (or amount) of the one or more VCs,
the presence or absence of other VCs in combination,
the ratio amounts between the various VCs, and
a change in the presence or amount of one or more VCs over time.

In methods and systems of the invention, and as disclosed hereinbelow, a pattern recognition module or analyzer is used to generate signal patterns that are characteristics of the VC profile (materials, amounts, ratios, etc). For the purpose of defining a VC profile or a signature indicative of VCs associated with presence or onset of the disease, knowing the nature and amount of the VCs is not necessary.

In most general terms, however, sensors utilized according to the invention are configured to interact and respond to the presence of VCs indicative of the onset, presence or evolution of NAFLD or BE, which may be Limonene (D-Limonene), Isoprene, Alpha-pinene, α-terpinene, Styrene, Propene, 2-Nonene, 1-decene, 1-heptene, 1-octene, Benzene, 2-Butanone, 2-pentanone, Acetone, Acetophenone, 2-Octanone, Ethanol, Methanol, 2-Propanol, Indole, Dimethyl-sulfide, Hydrogen-sulfide, carbon-disulfide, Methylmercaptan, Methyl-amine, Trimethyl-amine (TMA), Methane, Ethane, Pentane, Octane, Tridecane, 3-methyl-hexane, acetic acid, propionic acid, Acetaldehyde and others.

In some embodiments, a VC profile indicative of the onset, presence and progression of NAFLD or BE is based on the presence of 1, 2, 3, 4, 5, 6, 7 or 8 VCs. In some embodiments, the ratio between any two VCs in a combination of two or more VCs may be between 0.0001:1 and 1:0.0001. For example, in cases where the VC profile comprises two VCs, e.g., VC-A and VC-B, the ratio between the two may be between 0.0001:1 and 1:0.0001. In cases where three VOCs are present, e.g., VC-A, VC-B and VC-C, the ratio between VC-A and VC-B, the ratio between VC-A and VC-C and the ratio between VC-B and VC-C may be each between 0.0001:1 and 1:0.0001.

The electronic signal or pattern defining a VC profile indicative of the onset, presence or progression of a medical condition, as disclosed herein, is compared with an electronic signal or pattern defining a “control” sample or a plurality of samples, which may be characteristic of (i) a healthy subject population, namely a population that is not diseased, (ii) a population of subjects who have been tested and found not to have or suffer from the tested for disease—this population being regraded herein as the “negative group”, and/or (iii) a population of subjects who are suffering from the disease—herein rerefer to as the “positive group”. When comparing a signal indicative of the VC profile to a signal indicative to a VC profile of a positive and negative group, a determination can be made whether the subject has or does not have the disease or is developing the disease or is at an early stage of disease development (onset).

Control samples obtained for the purpose of determining the presence or absence or onset of the disease are typically taken from a plurality (one or more) of subjects which have been identified as healthy (not having the disease-thus a negative group) or as sick (who have contracted the diseases-thus a positive group). The number of subjects may be at least 10, 20, 30, 40, 50, 60, 70, 80, 90, 100, 150, 200, 250 to thousands of subjects.

Where determining progression of the disease, one or more VC profiles may be obtained for a group of subjects suffering from a disease, wherein each profile is obtained at a different time point along the way to recovery. Each profile may also be correlated to a different stage of treatment or recovery due to treatment.

Where the subject is a human, the control is a human, and where the method is used on non-human mammals, the control group should include species from the same group.

The invention further provides a method of determining presence, evolution (or development of early onset) and/or progression of a disease or a condition relating to or associated with NAFLD or BE in a subject, the subject being diagnosed or suffering from the disease or the condition or has a predisposition of suffering thereform or is suspected of having the disease, the method comprising determining based on a volatile compound (VC) profile obtained from a breath sample collected from the subject, presence of one or more marker molecules indicative of the presence, onset, or progression of the disease or condition.

In some embodiments, methods of the invention are purposed for early detection of NAFLD or conditions which may develop therefrom, such conditions may or may not comprise cancers and cancer-related conditions.

In some embodiments, methods of the invention are purposed for early detection of BE or conditions which may develop therefrom, such conditions may or may not comprise cancers and cancer-related conditions.

As used herein, “early detection” or detection of “onset of a disease” generally relates to capabilities of a device and methods of the invention for detecting sub-ppm levels of VCs or VOCs which are generated early on and thus which detection is now made possible before the diseases can be reliably detected through conventional standard-of-care/surveillance monitoring methods such as blood tests, imaging and others. In some cases, early detection may be achieved or confirmed by monitoring serially collected samples at a plurality of time points for the presence of the VCs or VOCs, as described herein.

In some embodiments, the method comprises obtaining a breath sample from a subject by employing any non-invasive means known in the art.

The invention further provides a device for determining a content profile of at least one VC in a gaseous sample, e.g., a breath sample obtained from a subject, the device comprising an array of sensors and a pattern-recognizer, and is configured for collection the gaseous sample in one or more sampling units, chambers or devices, configured to receive and hold a volume of the gaseous sample to be evaluated.

The device may be configured as a handheld device, as a partially disposable device and/or as a device for immediate or on-the-spot real-time analysis of VC sample content.

In some embodiments, the device comprises:
means for collecting the sample, e.g., a breath sample;
at least one sample collecting chamber, which may allow capturing the sample;
at least one sensor assembly; and
at least one communication means operable to direct said breath sample from the means for collecting the breath sample through the inlet to the at least one sample collecting chamber, and/or the at least one sensor assembly.

The device may further comprise a data processing unit for data communication with the sensor(s) in the sensor assembly; a data user interface unit being in data communication with the data processing unit; wherein the data processing unit comprising data relating to a control data set and is adapted to receiving from the sensor(s) information relating to presence of VCs or pattern thereof and provide an indication of presence or absence of one or more VCs, toxic material, disease state and other parameters as may be required.

The sensor utilized for purposes herein may be any device for measuring/detecting components of exhaled breath for achieving a determination of a profile of a breath sample as described herein. Some non-limiting examples of sensors that can be used in accordance with the present invention includes functionalized surface regions (wherein such surfaces are functionalized with metal nanoparticles, functional molecules, hollow fibers and others), sensors having a functionalized nanowire or a nanotube, a polymer-coated surface acoustic wave (SAW) sensors, sensor employing a semiconductor gas sensor technology, aptamer biosensors, amplifying fluorescent polymer (AFP) sensors and others.

In accordance with the present invention, the sensor may commercially be referred to as an “artificial nose” or as an “electronic nose” which can non-invasively measure at least one VC in the exhaled breath and/or monitor the concentration of at least one VC in the exhaled breath of a subject as described herein. Thus, the herein described sensors enable qualitative and/or quantitative analysis of volatile compounds (e.g., gases, vapors, or odors) hence facilitates the device to carry out a method of the invention.

In some embodiments, the device comprises one or more (or an array) of chemically sensitive sensors and a processing unit comprising a learning and pattern recognition analyzer configured for receiving sensor output signals and comparing the signals to a stored data, by utilizing a pattern recognition algorithm.

In some embodiments, the device may be a device disclosed in International Publication No. WO 2009/144725, herein incorporated by reference.

In some embodiments, the device utilizes a sensor as disclosed in US 2011/0269632, herein incorporated by reference.

As disclosed herein, the sensors are not, nor comprise, microbalances or metal oxides.

In some embodiments, the sensor is provided in the form of a plurality of nanoparticles that are associated to a surface. The sensor surface may comprise one or more sensing regions, each of the regions being associated with same or different population of nanoparticles, such that a signal may be independently derived from each of the sensing areas, and be indicative of an interaction (or lack thereof) between VCs present in the sample and the nanoparticles on the sensing regions.

Each of the sensing regions present on the sensor surface comprises a plurality of nanoparticles of a particular population, wherein each population differs from another in at least one of particle size, particle morphology (e.g., core/shell particles, non-core/shell particles, spherical, cubic, tetrahedral, triangular, dumbbell, elongated, multiparticles or fused particles, etc), particle composition (e.g., doping, metallic particles, non-metallic particles, conductive particles, novel metal particles, hybrid materials, etc), surface decoration (e.g., presence of material islands, association with ligand groups, etc) and others.

In some embodiments, each sensing region comprises a different selection of nanoparticles. In some embodiments, each sensing region comprises a mixed population (an inhomogeneous population) of nanoparticles, while in other embodiments, each sensing region comprises a uniform population (a homogenous population) of nanoparticles.

In a plurality of such sensing regions, one or more thereof may comprise a plurality of particle populations, namely an inhomogeneous population of particles, wherein some of the nanoparticles differ in structure, others in composition and still others in surface decoration. For example, a sensing region may comprise two populations of nanoparticles, one population comprising particles of one metal and another population comprises particles of a different metal. In a similar way, all particles may be of one metal but differ from each other in their surface decoration (e.g., presence of ligands or selection of ligands).

In some embodiments, the nanoparticles are core/shell particles, non-core/shell particles, spherical, cubic, tetrahedral, triangular, dumbbell, elongated or fused particles. In some embodiments, the particles are spherical in shape.

In some embodiments, the nanoparticles are metallic nanoparticles; wherein the metal is optionally selected amongst any metal of the Periodic Table of the Elements. In some embodiments, the metals are of any of Groups IIIB, IVB, VB, VIB, VIIB, VIIIB, IB and IIB of block d of the Periodic Table. In some embodiments, the metal is selected from Sc, Ti, V, Cr, Mn, Fe, Ni, Cu, Y, Zr, Nb, Tc, Ru, Mo, Rh, W, Au, Pt, Pd, Ag, Au, Al, Mn, Co, Cd, Hf, Ta, Re, Os, Ir and Hg.

In some embodiments, the metal is gold, silver, nickel, cobalt, copper, palladium, platinum or aluminum. In some embodiments, the nanoparticles are gold nanoparticles.

In some embodiments, the metal is not in a form of a metal oxide.

The metallic nanoparticles may or may not be doped or further comprise an amount of another metallic or non-metallic material. The metallic nanoparticles may be bare, namely uncoated, or coated with a plurality of surface associated ligand molecules. Such ligand molecules may have surface anchoring groups which may vary based on, e.g., the composition of the nanoparticles. For example, where the nanoparticles are gold nanoparticles, the surface anchoring groups may be a thiol, a disulfide, an amine and others as known in the art.

In some embodiments, the metallic nanoparticles were provided with a coating of ligand molecules. Coating was achieved as known in the art.

The ligand molecules, typically organic molecules, were selected from various types of thiols, disulfides, and amine-containing molecules. Such ligand molecules were butanethiol, hexanethiol, 1-heptanethiol, decanethiol, dodecanethiol, tert-dodecanethiol, 2-ethylhexanethiol, 4-tert methylbenzenethiol, dibutyl disulfid, 3-ethpxythiolphenol, 4-chlorobenzenemethanethiol, 2-nitro-4-(trifluoromethyl)benzenethiol, benzylmercaptan, 4-mercaptophenol, 4-mercaptobenzonitrile, 3-mercapto-1-propanol, 4-mercaptobutan-1-ol, and 6-mercapto-1-hexanol.

Different gold nanoparticle populations were formed from using one or more of the ligands disclosed herein or different mixtures of these ligands.

The invention further provides a method of using a device according to the invention, the method comprising obtaining a breath sample from a subject by employing any non-invasive means known in the art and directing or permitting flow of said breath sample into the device of the invention.

Determining a VC profile or a profile of a breath sample obtained from a subject permit also management and monitoring of treatment protocols. Such management of treatment protocols may include one or more of
Patients' stratification for treatment with therapies that are effective mainly in specific disease phenotypes (companion diagnostics);
Ability to identify patients who are most likely to benefit from a particular therapeutic product;
Prediction of drug-induced toxicity in order to provide advanced warning of potential side effects;
Identification of patients likely to be at increased risk for serious side effects as a result of treatment with a particular therapeutic product;
Monitoring disease progression and response to treatment to enable treatment adjustments to achieve improved safety or effectiveness.

More so, management of treatment protocols involving the use of a methodology or a device of the invention has the additional benefit of monitoring a patient's response to treatment, which can be used to help optimize treatment doses for the subject's own metabolism.

Thus, in another aspect, the invention provides a method for evaluating or determining treatment response and/or drug-induced toxicity in a subject who has undergone or who is undergoing a medical treatment, the process comprises utilizing a method of the invention for determining presence or absence of one or more VCs indicative of an evolving drug-induced toxicity.

The invention further provides a method for evaluating drug related volatiles, e.g., for determining the drug's pharmacokinetics and/or pharmacodynamics, the method comprising utilizing a method of the invention for determining presence or absence of one or more drug-related volatile. The breath VOC profile includes VOCs produced during the metabolism of pharmaceuticals and other xenobiotics. Observing changes in the type and concentration of metabolites over time after the administration of pharmaceuticals is a useful tool in drug pharmacokinetics (and drug compliance studies), enabling changes in levels of metabolites to be investigated noninvasively via breath. Thus, the invention is further purposed for determining presence and/or concentration of drug related volatiles indicative of a drug's metabolism in the subject, during treatment of said subject with the drug.

The utilization of methods of the invention may allow for evaluation of microbiome associated with treatment response or side effects (e.g., a specific microbiome known to be associated with a response to immunotherapy); assessment of metabolic rates for the CYP450 liver enzymes, which influence the efficacy of many common drugs; drug treatments and disease progression which may result in changes in volatile metabolites which can be detected in the breath.

Breath biomarkers are suitable for use as companion diagnostics to screen and stratify patients for inclusion in clinical trials, by selection of patients most likely to respond to a particular therapy. Consequently, clinical trials have a greater likelihood of success of showing good efficacy for therapies that are effective mainly in specific disease phenotypes.

VC biomarkers can be identified to predict therapy response and treatment efficacy. It can lead to the development of diagnostic breath tests for identification of responders.

Comparing pre- and post-treatment breath tests could provide confirmation of therapy efficacy by detecting changes in VOCs profile. Furthermore, a change in breath VOCs profile may provide the first signs of changes in the underlying disease state, as diseases progress or adapt to resist treatment.

Methods of the invention may additionally be used to detect onset, disease state or progression of a disease state which fatty liver (or NAFLD) or BE may be a precursor to such a disease state. Such disease states may be inflammation and cancers as disclosed herein.

In another aspect, the invention provides a method for determining onset or early detection of NAFLD, the method comprises two main steps:
1) Administering to a subject suspected of suffering from NAFLD or having predisposition for suffering from the disease or any subject for whom such a determination is to be made, at least one material being: (i) a material known to undergo degradation or chemical modification, e.g., via an enzymatic reaction, that is unique to or indicative of or relating to NAFLD, or (ii) a material known to undergo degradation or chemical modification, e.g., via an enzymatic reaction, that is unique to or indicative of or relating to NAFLD at a slower or greater rate as compared to such degradation or modification in subjects not having NAFLD, or (iii) a material known not to undergo any degradation or modification due to NAFLD-related changes in or deactivation of at least one degradation pathway; and
2) Detection of a VC profile using methods as disclosed herein.

The administration of the at least one material may be used to more specifically and with higher sensitivity to detect a presence and/or amount of a predetermined material (VC or VOC). For example, in some embodiments, the method may comprise administering to the subject limonene (alone or provided as a food product or a food or drink formulation). As stated hereinabove, differences between control and diseased subjects observed in several studies reflect differences in the activity of the CYP450 enzyme system, reduction of liver functional units, reduced portal blood flow, and impaired liver extraction capacity. NAFLD involves downregulation of the CYP450 system which includes the limonene metabolizing enzymes. As a result, greater amounts of limonene are expected to remain unmetabolized or unprocessed. Thus, greater amounts of limonene are expected to be excreted to the circulatory system and subsequently exit the body through, e.g., the breath. Breath samples obtained from a subject having a reduced activity of the CYP450 due to development of NAFLD are expected to contain higher concentrations of limonene, as compared to samples obtained from a healthy subject. Administration of limonene, e.g., through oral delivery of a food product or a drink enriched with a predetermined amount of limonene, can improve diagnostic evaluation of the disease state and disease onset.

In some embodiments, a method of the invention thus further comprises administering to the subject limonene and carrying out a method as disclosed herein for detecting a limonene profile in a breath sample obtained from the subject.

The invention further provides a method comprising prior to determining a volatile compound (VC) profile in a breath sample, as disclosed herein, administering to the subject: (i) a material known to undergo degradation or chemical modification, that is unique to or indicative of or relating to NAFLD, or (ii) a material known to undergo degradation or chemical modification, that is unique to or indicative of or relating to NAFLD, and which degradation is at a slower or greater rate as compared to such degradation or modification in subjects not having NAFLD, or (iii) a material known not to undergo a degradation or modification due to NAFLD-related changes in or deactivation of at least one degradation pathway.

Also provided is a method of determining a reduction or a decline in activity or level of cytochrome P450 (CYP450) in a subject, the subject being diagnosed or suffering from a disease or a condition or has a predisposition of suffering from a disease or condition associated with a reduction or a decline in CYP450 activity, the method comprising:
a) administering to the subject a formulation comprising limonene;
b) determining a volatile compound (VC) profile in a breath sample collected from the subject, wherein the breath sample comprises limonene; and
c) comparing the VC profile to a VC profile of a control comprising limonene, and/or optionally to a VC profile obtained from the subject at an earlier time point(s);
wherein a VC profile indicative of presence of greater amounts of limonene as compared to the control VC profile or to the VC profile obtained from the subject at an earlier time point is indicative of a reduction in the activity of the CYP450.

The reduction or decline in CYP450 activity is typically such which decreases degradation or metabolization of endogenous and exogenous chemicals, such as potentially toxic compounds, including drugs and products of endogenous metabolism, principally in the liver. Such a reduction may be quantified by a variety of analytical tools available in the medical arena. As example herein, the reduction or decline in CYP450 activity is reflected in a reduction in the metabolism of limonene as compared to a CYP450 system of normal activity (a control). As compared to the control, the reduction may be reflected in an increase in the amounts of limonene found in the breath sample obtained from a subject having a deficiency in the CYP40 system or a reduction in its activity. As compared to the control, a measurable reduction may be reflected in a minimum increase of anywhere between 5 and 25% in signal or VC profile associated with limonene in the breath sample.

The reduction in the enzyme activity may suggest onset of NAFLD. Thus, the invention further provides method for determining presence, evolution (or onset) and/or progression of NAFLD in a subject, the subject being diagnosed or suffering from the disease or the condition or has a predisposition of suffering therefrom, the method comprising:
a) administering to the subject a formulation comprising limonene;
b) determining a volatile compound (VC) profile in a breath sample collected from the subject, wherein the breath sample comprises limonene; and
c) comparing the VC profile to a VC profile of a control comprising limonene, and/or optionally to a VC profile obtained from the subject at an earlier time point(s);
wherein a VC profile indicative of a different amount or level of limonene as compared to the control VC profile is indicative of one or more of (1) presence the NAFLD condition, (2) absence of the NAFLD condition, or (3) an improvement or worsening of the NAFLD.

The invention further provides:

In some embodiments of methods of the invention, the invention concerns a method for determining presence, onset and/or progression of non-alcoholic fatty liver disease (NAFLD) in a subject, the subject being diagnosed with or suffering from the disease or has a predisposition of suffering thereform, the method comprising:
a) determining a volatile compound (VC) profile of a breath sample collected from the subject; and
b) comparing the VC profile to a VC profile of a control, and/or optionally to a VC profile obtained from the subject at an earlier time point(s), or from another subject who has been determined healthy or carrying the disease;
wherein a VC profile different from the control VC profile, or the VC profile obtained from the subject or from the another subject is indicative of one or more of (1) presence of the NAFLD disease, (2) absence of the NAFLD disease, or (3) an improvement or worsening of the NAFLD disease.

In some embodiments of methods of the invention, the method is for determining a reduction in cytochrome P450 (CYP450) activity or level indicative of NAFLD onset.

In some embodiments of methods of the invention, the invention provides a method of determining a reduction or a decline in activity or level of cytochrome P450 (CYP450) in a subject, the subject being diagnosed or suffering from a disease or a condition or has a predisposition of suffering from a disease or a condition associated with a reduction or a decline in CYP450 activity, the method comprising:
a) determining a volatile compound (VC) profile in a breath sample collected from the subject; and
b) comparing the VC profile to a VC profile of a control, and/or optionally to a VC profile obtained from the subject at an earlier time point(s), or from another subject who has been determined healthy or carrying the disease;
wherein a VC profile different from the control VC profile, or the VC profile obtained from the subject or from the another subject is indicative of a reduction in the activity of the CYP450.

In some embodiments of methods of the invention, the subject is diagnosed with or is suffering from or has a predisposition of suffering from non-alcoholic fatty liver disease (NAFLD).

In some embodiments of methods of the invention, the VC profile obtained from the subject is different from the control VC profile, or the VC profile obtained from the subject or from the another subject in presence or increased levels of limonene.

In some embodiments of methods of the invention, wherein determining a volatile compound (VC) profile in a breath sample comprises flowing the breath sample over an array of sensors, each of said array of sensors comprises a plurality of nanoparticles capable of interacting with volatile compounds present in the breath sample to generate an electric signal; said electric signal being different from that obtained for a control sample not comprising the volatile compounds or comprising different amounts or ratios of the volatile compounds.

In some embodiments of methods of the invention, the nanoparticles are gold nanoparticles.

In some embodiments of methods of the invention, the gold nanoparticles are coated with one or more types of organic ligands.

In some embodiments of methods of the invention, the organic ligands are selected from hexanethiol, 1-heptanethiol, decanethiol, dodecanethiol, tert-dodecanethiol, 2-ethylhexanethiol, 4-tert methylbenzenethiol, dibutyl disulfid, 3-ethpxythiolphenol, 4-chlorobenzenemethanethiol, 2-nitro-4-(trifluoromethyl)benzenethiol, benzylmercaptan, 4-mercaptophenol, 4-mercaptobenzonitrile, 3-mercapto-1-propanol, 4-mercaptobutan-1-ol, and 6-mercapto-1-hexanol.

In some embodiments of methods of the invention, the organic ligands is a combination of two or more organic ligands selected from hexanethiol, 1-heptanethiol, decanethiol, dodecanethiol, tert-dodecanethiol, 2-ethylhexanethiol, 4-tert methylbenzenethiol, dibutyl disulfid, 3-ethpxythiolphenol, 4-chlorobenzenemethanethiol, 2-nitro-4-(trifluoromethyl)benzenethiol, benzylmercaptan, 4-mercaptophenol, 4-mercaptobenzonitrile, 3-mercapto-1-propanol, 4-mercaptobutan-1-ol, and 6-mercapto-1-hexanol.

In some embodiments of methods of the invention, the organic ligand is hexanethiol.

In some embodiments of methods of the invention, the organic ligand is 1-heptanethiol.

In some embodiments of methods of the invention, the organic ligand is decanethiol.

In some embodiments of methods of the invention, the organic ligand is dodecanethiol.

In some embodiments of methods of the invention, the organic ligand is tert-dodecanethiol.

In some embodiments of methods of the invention, the organic ligand is 2-ethylhexanethiol.

In some embodiments of methods of the invention, the organic ligand is 4-tert methylbenzenethiol.

In some embodiments of methods of the invention, the organic ligand is dibutyl disulfide.

In some embodiments of methods of the invention, the organic ligand is 3-ethpxythiolphenol.

In some embodiments of methods of the invention, the organic ligand is 4-chlorobenzenemethanethiol.

In some embodiments of methods of the invention, the organic ligand is 2-nitro-4-(trifluoromethyl)benzenethiol.

In some embodiments of methods of the invention, the organic ligand is benzylmercaptan.

In some embodiments of methods of the invention, the organic ligand is 4-mercaptophenol.

In some embodiments of methods of the invention, the organic ligand is 4-mercaptobenzonitrile.

In some embodiments of methods of the invention, the organic ligand is 3-mercapto-1-propanol.

In some embodiments of methods of the invention, the organic ligand is 4-mercaptobutan-1-ol.

In some embodiments of methods of the invention, the organic ligand is 6-mercapto-1-hexanol.

In some embodiments of methods of the invention, prior to determining the volatile compound (VC) profile in the breath sample, administering to the subject: (i) a material known to undergo degradation or chemical modification, that is unique to or indicative of or relating to NAFLD, or (ii) a material known to undergo degradation or chemical modification, that is unique to or indicative of or relating to NAFLD, and which degradation is at a slower or greater rate as compared to such degradation or modification in a subject not having NAFLD, or (iii) a material known not to undergo a degradation or modification caused by a NAFLD-related change in or deactivation of at least one degradation pathway.

In some embodiments of methods of the invention, prior to determining the volatile compound (VC) profile in the breath sample, administering to the subject a material known to undergo degradation or chemical modification, that is unique to or indicative of or relating to NAFLD, and which degradation is at a slower or reduced as compared to such degradation or modification in a subject not having NAFLD.

In some embodiments of methods of the invention, the material is limonene.

In some embodiments of methods of the invention, the method is for determining presence, onset and/or progression of non-alcoholic fatty liver disease (NAFLD) by determining a reduction or a decline in activity or level of cytochrome P450 (CYP450) in a subject, the subject being diagnosed with or suffering from NAFLD or has a predisposition of suffering therefrom, the method comprising:
a) administering to the subject a formulation comprising limonene;
b) determining a volatile compound (VC) profile in a breath sample collected from the subject, wherein the breath sample comprises limonene; and
c) comparing the VC profile to a VC profile of a control comprising limonene, and/or optionally to a VC profile obtained from the subject at an earlier time point(s) or from another subject having been determined healthy or suffering from a reduced CYP450 activity;
wherein where the VC profile is indicative of presence of greater amounts of limonene as compared to the control VC profile or to the VC profile obtained from the subject or from the another subject is indicative of a reduction in the activity of the CYTP450 and presence or onset of NAFLD.

In some embodiments of methods of the invention, the subject does not exhibit symptoms of NAFLD.

In some embodiments of methods of the invention, the detectable level of limonene in the beath sample is parts per billion (ppb).

In some embodiments of methods of the invention, the limonene formulation is a food or a drink comprising limonene and administered by consumption.

In some embodiments of methods of the invention, the breath sample is collected from the subject administered with the formulation 20 to 60 minutes following administration. In some embodiments of methods of the invention, the sample is collected within a period of 20, 60 minutes from administration, or between 20 and 50 minutes, or between 20 and 40 minutes or between 20 and 30 minutes from administration. In some embodiments of methods of the invention, the sample is collected within a period of 10 and 20 minutes, or within 10 and 30 minutes from administration.

In some embodiments of methods of the invention, the method is a method for immediate or on-the-spot determining of onset or presence of NAFLD.

In some embodiments of methods of the invention, the method is for determining a disease state in the subject, wherein the disease state is severe steatohepatitis (NASH), liver fibrosis, cirrhosis, or hepatic carcinoma.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 depicts different sensor response to different biomarker concentrations.

FIG. 2 provides results of a PCA analysis used to generate un-supervised data clusters, classified by LDA to differentiate samples spiked and not spiked with the biomarker.

FIG. 3 depicts a sensor sensitivity and selectivity.

### DETAILED DESCRIPTION OF EMBODIMENTS

### Example 1: Sensor Array

Sensor array used in detection of VC profiles for determining NAFLD were structured of chemiresistive layers of spherical gold nanoparticles (GNPs, core diameter: 2-20 nm) coated with one or more type of organic residues or ligands. The organic residues were associated to the surface of the GNP by utilizing methodologies known in the art.

Successful determination of NAFLD onset was achieved by use of GNP coated with organic residues such as butanethiol, hexanethiol, 1-heptanethiol, decanethiol, dodecanethiol, tert-dodecanethiol, 2-ethylhexanethiol, 4-tert methylbenzenethiol, dibutyl disulfid, 3-ethpxythiolphenol, 4-chlorobenzenemethanethiol, 2-nitro-4-(trifluoromethyl)benzenethiol, benzylmercaptan, 4-mercaptophenol, 4-mercaptobenzonitrile, 3-mercapto-1-propanol, 4-mercaptobutan-1-ol, and 6-mercapto-1-hexanol. Different nanoparticle populations were used, wherein each population was formed with a different organic ligand or with a different mixture of ligand types. The selection of different organic ligands provided broad cross-reactive absorption sites for VOCs.

The GNP chemiresistive layers are formed on a microelectronic transducer.

The sensor array composed of GNPs with the different coatings was provided in a hand-held/small portable device with sensor exposure chamber in which the VOC samples was allowed to flow. The device comprised two detachable parts enabling easy replacement of the sensor chamber. The sensor array was connected to a circuit board, enabling sensors resistance measurement over time.

Sensor resistance was recorded during baseline-before exposure, during exposure to tested samples, and during sensor cleaning.

### Example 2: Sensor Sensitivity to NAFLD Biomarkers (e.g., Limonene)

Different sensors formed of gold nanoparticles coated with ligand molecules were tested to study sensor sensitivity. The results shown below are for mixed sensor populations comprising two or more types of GNP, each type comprising a different ligand coating. The ligands used were: hexanethiol, 1-heptanethiol, decanethiol, dodecanethiol, tert-dodecanethiol, 2-ethylhexanethiol, 4-tert methylbenzenethiol, dibutyl disulfid, 3-ethpxythiolphenol, 4-chlorobenzenemethanethiol, 2-nitro-4-(trifluoromethyl)benzenethiol, benzylmercaptan, 4-mercaptophenol, 4-mercaptobenzonitrile, 3-mercapto-1-propanol, 4-mercaptobutan-1-ol, and 6-mercapto-1-hexanol.

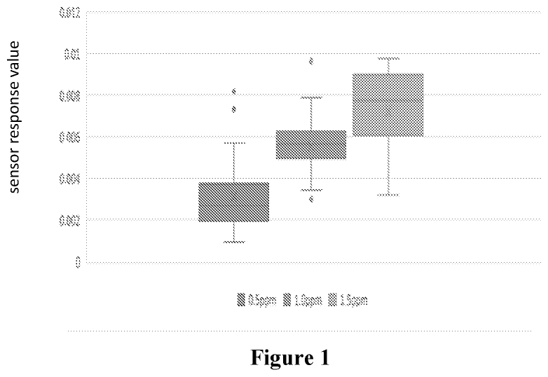

Sensors were exposed to different concentrations of NAFLD biomarkers being or including limonene, wherein the marker concentration was between 500 ppb and 1.5 ppm. Smaller concentrations and higher concentrations were also separately tested. The different sensor response to different biomarker concentrations is shown in FIG. 1. 31 sensor units were tested. Each box represents the middle 50% of observed values. The bottom of the box is the first quartile (25th percentile) and the top of the box is the third quartile (75th percentile). The line in the middle of the box is the median (50th percentile), and X represents the average response. STD and outliers are also presented.

The results support the initial observations according to which sensor sensitivity to markers of NAFLD found in breadth samples was high. Marker concentrations as low as 100-500 ppb could be measured and could be used to distinguish between test and control samples. Detection of such low concentrations permit detection of limonene in breath samples obtained from patients demonstrating onset of NAFLD.

### Example 3: Classification Between Breath Samples without and with Spike of NAFLD Biomarker

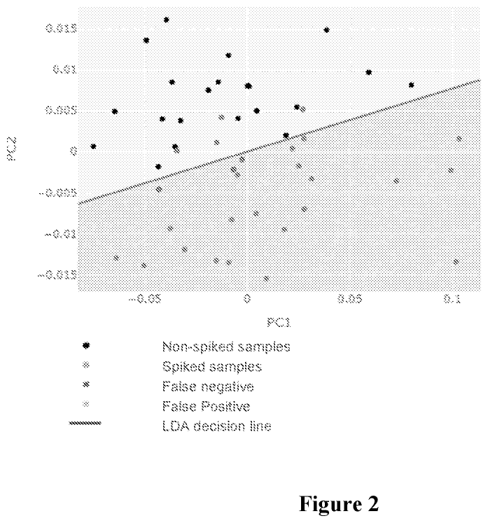

48 breath samples from healthy volunteers were collected. 24 samples were spiked with NAFLD volatile biomarkers, such as limonene. Samples were then exposed to a sensor of the invention. Each exposure resulted in timeseries vectors of resistance measurement points. PCA analysis was used to generate un-supervised clusters, that were then classified by LDA to differentiate samples spiked and not spiked with the biomarker (FIG. 2).

Classification performance was assessed by 6-fold Cross validation-results of the train and test groups are shown in Table 1.

TABLE 1

| | Whole Dataset | Cross validation * |
|---|---|---|
| Accuracy | 85% | 85% |
| Sensitivity | 83% | 83% |
| Specificity | 88% | 88% |

As may be understood, the experimental model allows to recreate the physiological characteristics of a NAFLD patient's breath. These results show overall accuracy of about 85% with cross validation methodology.

### Example 4: Classification Between Breath Samples of Heathy Volunteers and Subject Diagnosed with NAFLD

Breath samples were collected from healthy volunteers and NAFLD subjects and exposed to a sensor according to the invention. Each exposure resulted in timeseries vectors of resistance points. Each vector was composed of 172 resistance measurement points. 94 vectors were collected. A Random Forest model was developed to assess the classification between NAFLD and healthy samples.

The results of 5-fold cross validation (75 train\19 test) are shown in Table 2.

Classification between groups with 93.62 accuracy demonstrated sensitivity of 91% and specificity of 96%.

TABLE 2

| Timepoint [min] | (%) |
|---|---|
| Accuracy | 93.62 |
| Sensitivity | 91.11 |
| Specificity | 96 |

### Example 5: Classification Between Breath Samples of Heathy Volunteers and Subject Diagnosed with NAFLD, Prior to and Following Limonene Administration

As explained above, dietary VOCs are processed by hepatic CYP450 enzymes. In fatty liver, due to the decrease in CYP450 activity there is an increase in dietary VOCs found in breath. Orange juice includes high concentration of several VOCs. Breath samples from healthy volunteers and NAFLD subjects were collected prior to and in different timepoints (10 min and 20-30 min) following consumption of orange juice containing limonene. Sample were then exposed to the sensor. Each exposure resulted in timeseries vectors of resistance measurement points. Each vector is composed of 172 resistance measurement points.

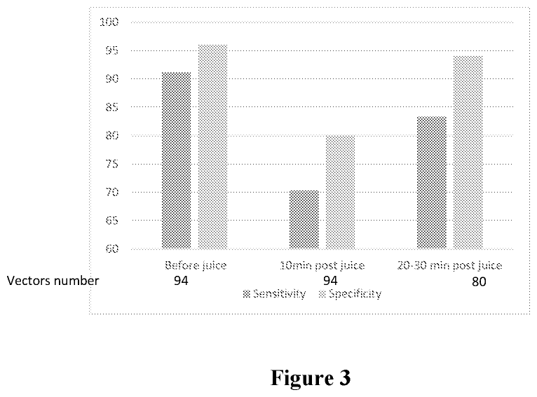

For each timepoint multiple vectors were collected (number in each timepoint is indicated in FIG. 3).

A Random Forest model was developed to assess the performance for each time point separately. The sensitivity and specificity calculated by 5-fold cross validation are presented in the following graphs.

Before orange juice administration, the sensors classified between healthy and NAFLD breath samples with 91% sensitivity. 10 min following orange juice administration, sensitivity was significantly reduced probably due to the excess of orange-juice-originated VOCs that were not metabolized by the hepatic enzymes, both in the healthy and fatty liver, and therefore released in breath in both groups. However, 20-30 min following the orange juice administration, after the excess VOCs were released, sensitivity of the test is increased.

1.-37. (canceled)

38. A method for determining presence, onset and/or progression of non-alcoholic fatty liver disease (NAFLD) in a subject, the subject being diagnosed with or suffering from the disease or has a predisposition of suffering thereform, the method comprising:
a) determining a volatile compound (VC) profile of a breath sample collected from the subject, wherein determining a volatile compound (VC) profile in a breath sample comprises flowing the breath sample over an array of sensors, each of said array of sensors comprises a plurality of nanoparticles capable of interacting with volatile compounds present in the breath sample to generate an electric signal; said electric signal being different from that obtained for a control sample not comprising the volatile compounds or comprising different amounts or ratios of the volatile compounds; and
b) comparing the VC profile to a VC profile of a control, and/or optionally to a VC profile obtained from the subject at an earlier time point(s), or from another subject who has been determined healthy or carrying the disease;
wherein a VC profile different from the control VC profile, or the VC profile obtained from the subject or from the another subject is indicative of one or more of (1) presence of the NAFLD disease, (2) absence of the NAFLD disease, or (3) an improvement or worsening of the NAFLD disease.

39. The method according to claim 38, for determining a reduction in cytochrome P450 (CYP450) activity or level indicative of NAFLD onset.

40. A method of determining a reduction or a decline in activity or level of cytochrome P450 (CYP450) in a subject, the subject being diagnosed or suffering from a disease or a condition or has a predisposition of suffering from a disease or a condition associated with a reduction or a decline in CYP450 activity, the method comprising:
a) determining a volatile compound (VC) profile in a breath sample collected from the subject; and
b) comparing the VC profile to a VC profile of a control, and/or optionally to a VC profile obtained from the subject at an earlier time point(s), or from another subject who has been determined healthy or carrying the disease;
wherein a VC profile different from the control VC profile, or the VC profile obtained from the subject or from the another subject is indicative of a reduction in the activity of the CYP450.

41. The method according to claim 40, wherein the subject is diagnosed with or is suffering from or has a predisposition of suffering from non-alcoholic fatty liver disease (NAFLD).

42. The method according to claim 38, wherein the VC profile obtained from the subject is different from the control VC profile, or the VC profile obtained from the subject or from the another subject in presence or increased levels of limonene.

43. The method according to claim 38, wherein the nanoparticles are gold nanoparticles, optionally coated with one or more types of organic ligands.

44. The method according to claim 38, wherein prior to determining the volatile compound (VC) profile in the breath sample, administering to the subject: (i) a material known to undergo degradation or chemical modification, that is unique to or indicative of or relating to NAFLD, or (ii) a material known to undergo degradation or chemical modification, that is unique to or indicative of or relating to NAFLD, and which degradation is at a slower or greater rate as compared to such degradation or modification in a subject not having NAFLD, or (iii) a material known not to undergo a degradation or modification caused by a NAFLD-related change in or deactivation of at least one degradation pathway.

45. The method according to claim 44, wherein prior to determining the volatile compound (VC) profile in the breath sample, administering to the subject a material known to undergo degradation or chemical modification, that is unique to or indicative of or relating to NAFLD, and which degradation is at a slower or reduced as compared to such degradation or modification in a subject not having NAFLD.

46. The method according to claim 45, wherein the material is limonene.

47. The method according to claim 38, for determining presence, onset and/or progression of non-alcoholic fatty liver disease (NAFLD) by determining a reduction or a decline in activity or level of cytochrome P450 (CYP450) in a subject, the subject being diagnosed with or suffering from NAFLD or has a predisposition of suffering therefrom, the method comprising:
a) administering to the subject a formulation comprising limonene;
b) determining a volatile compound (VC) profile in a breath sample collected from the subject, wherein the breath sample comprises limonene; and
c) comparing the VC profile to a VC profile of a control comprising limonene, and/or optionally to a VC profile obtained from the subject at an earlier time point(s) or from another subject having been determined healthy or suffering from a reduced CYP450 activity;
wherein where the VC profile is indicative of presence of greater amounts of limonene as compared to the control VC profile or to the VC profile obtained from the subject or from the another subject is indicative of a reduction in the activity of the CYTP450 and presence or onset of NAFLD.

48. The method according to claim 38, wherein the subject does not exhibit symptoms of NAFLD.

49. The method according to claim 48, wherein the detectable level of limonene in the beath sample is parts per billion (ppb).

50. The method according to claim 48, wherein the limonene formulation is a food or a drink comprising limonene and administered by consumption.

51. The method according to claim 48, wherein the breath sample is collected from the subject administered with the formulation 20 to 60 minutes following administration.

52. The method according to claim 38, being a method for immediate or on-the-spot determining of onset or presence of NAFLD.

53. The method according to claim 38, for determining a disease state in the subject, wherein the disease state is severe steatohepatitis (NASH), liver fibrosis, cirrhosis, or hepatic carcinoma.
